(19) United States
(12) Patent Application Publication
(10) Pub. No.: US 2013/0165819 A1
(43) Pub. Date: Jun. 27, 2013

(54) System and method for controlling radio frequency scanning attributes of an implantable medical device

(71) Applicant: Thanh Tieu, Simi Valley, CA (US)

(72) Inventor: Thanh Tieu, Simi Valley, CA (US)

(73) Assignee: PACESETTER, INC., Sylmar, CA (US)

(21) Appl. No.: 13/336,725

(22) Filed: Dec. 23, 2011

(51) Int. Cl. A61B 5/11

(52) U.S. Cl. 600/595

(57) ABSTRACT

A method and system are provided for controlling radio frequency (RF) scanning attributes of an implantable medical device (IMD) that include configuring an IMD to establish an RF connection over a predetermined frequency band based on a scan attribute. The method and system may also include storing, in the IMD, predetermined first and second values for the scan attribute to define different first and second scan modes, respectively. The method and system may also include determining a posture state of a patient, in which the IMD is implanted, and switching between the predetermined first and second values for the scan attribute, based on the posture state determined, to cause the IMD to switch between the first and second scan modes.

### BACKGROUND OF THE INVENTION

Embodiments of the present invention generally relate to a system and method for controlling radio frequency (RF) scanning attributes of an implantable medical device (IMD), and more particularly to system and method of adjusting the RF scanning attributes based on a detected position of an IMD within a patient.

Numerous medical devices exist today, including but not limited to electrocardiographs (“ECGs”), electroencephalographs (“EEGs”), squid magnetometers, implantable pacemakers, implantable cardioverter-defibrillators (“ICDs”), neurostimulators, electrophysiology (“EP”) mapping and radio frequency (“RF”) ablation systems, and the like (hereafter generally “implantable medical devices” or “IMDs”). IMDs commonly employ one or more leads with electrodes that either receive or deliver voltage, current or other electromagnetic pulses (generally “energy”) from or to an organ or tissue (collectively hereafter “tissue”) for diagnostic or therapeutic purposes.

Various IMDs are monitored by remote care or base stations that are remotely located from the IMDs. For example, a patient may have an IMD that communicates with a base station within the patient's home. The base station may be located by a patient's bedside. The base station receives data from the IMD about the patient's physiological state and/or the operation state of the IMD. Based on the received data, the base station may convey the data to a remote server of a medical care network, or adjust operating parameters for the IMD. For example, the base station may adjust operating parameters of the IMD, such as when a patient experiences changes in arrhythmia, pacing, ST shift, various types of ischemia, base rate, and the like.

Today, most IMDs include an RF capability to communicate with the base station. Data may be received from the base station when transmitted over varies frequency bands, such as at a 400-405 MHz frequency range.

The RF chip periodically scans select frequency bands, such as the 2.45 GHz band, over the life of the IMD. The IMD uses information received over the 2.45 GHz band to determine if the base station is seeking to communicate with the IMD over another band (for example, approximately 400 MHz band), which is used to receive and transmit data to and from the IMD. If the RF chip operating at a 2.45 GHz band detects that the base station desires to communicate over the 400 MHz band, the IMD then switches over to the 400 MHz band. Bidirectional communication over the 400 MHz band consumes substantially more power than the 2.45 GHz band.

Today, more and more patients have installed in their homes a portable patient care system (PCS), which functions as a base station that wirelessly communicates with the IMD. The PCS also communicates with a remote server within a patient care network. A scan protocol establishes a manner by which the IMD and PCS may initiate a communications session. As one example, the scan protocol provides that the IMD shall scan a first/high frequency range/band (e.g., 2.45 GHz band) continuously for connection requests. When a connection request is sensed in the first/high band, the IMD initiates a communications session with the PCS. The scan protocol further provides that the IMD shall scan a second/low frequency range/band (e.g., 400 MHz band) at a predetermined periodic interval (e.g., every 2 hours).

The first/high frequency band is used for “unscheduled” PCS follow up sessions, while the second/low frequency band is used for “scheduled” PCS sessions. An unscheduled session is initiated by the patent at any time by selecting a button on the PCS. The PCS transmits a connection request over the high frequency band. Because the IMD is scanning continuously over the high frequency band, the IMD senses the connection request, and initiates a PCS to IMD communications session.

A scheduled session is automatically requested by the PCS at certain times of day such as in the middle of the night while the patient sleeps. The PCS, with a low frequency wand therein is placed at all times near the bed. At predetermined times throughout the night, the PCS transmits (from the low frequency wand) connection requests over the low frequency band. The IMD scans the low frequency band at predetermined intervals (e.g., every 2 hours). If the PCS is transmitting a connection request for a scheduled session at the same time that the IMD scans the low frequency band, then the IMD will sense the connection request, and initiate a PCS to IMD communications session over the low frequency band.

The low frequency band affords a longer range and more robust connection than the high frequency band. However, the high frequency band draws less power from the IMD when scanning for connection requests and during a communications session.

However, conventional scan protocols exhibit certain limitations that lead to unnecessary depletion of battery life. For example, there is no need for the IMD to scan the high frequency band continuously in the middle of the night because it is very unlikely that a patient will seek to initiate an unscheduled session (that is, the patient will be asleep). Also, there is no need for the IMD to scan the low frequency band, even periodically, during the daytime because it is very unlikely that a patient will lay or sit next to the low frequency wand for a 2 hour period to catch the next scheduled connection request from the PCS.

### SUMMARY

Embodiments of the present invention provide an improved IMD and scan protocol that, among other things, overcomes the limitations discussed above.

Certain embodiments provide a method for controlling radio frequency (RF) scanning attributes of an implantable medical device (IMD) that includes configuring an IMD to establish an RF connection over a predetermined frequency band based on a scan attribute. The method may also include storing, in the IMD, predetermined first and second values for the scan attribute to define different first and second scan modes, respectively. The method may also include determining a posture state of a patient, in which the IMD is implanted, and switching between the predetermined first and second values for the scan attribute, based on the posture state determined, to cause the IMD to switch between the first and second scan modes.

The determining may include sensing a posture of a patient from a sensor within the IMD. The sensor may provide a posture signal and compare the posture signal to potential posture states to identify an actual posture state. The configuring may also include configuring the IMD to establish RF connections over primary and secondary frequency ranges that are non-overlapping and distinct from one another.

The scan attribute may represent scan rate. As such, the method may also include scanning the predetermined frequency band at different first and second rates when in the first and second scan modes, respectively. Optionally, the scan attribute may represent scan receive power, a frequency range of the predetermined frequency band, antenna impedance, antenna capacitance, and/or antenna inductance.

The method may also include calibrating the IMD to monitor for potential posture states including at least one of a supine state, prone state, right side position and left side position.

The method may also include scanning channels in the predetermined frequency band for an RF connection request from a base station. The method may also include, when in the first scan mode, scanning the predetermined frequency band at a first scan rate for an RF connection request and, when in the second scan mode, scanning the predetermined frequency band at a second scan rate for the RF connection request. The method may also include, in response to receiving an RF connection request over the predetermined frequency band, initiating scanning of a secondary frequency band for communications data from an external device.

Certain embodiments may provide an implantable medical device (IMD that includes an RF module configured to operate in first and second scan modes to establish an RF connection over a predetermined frequency band based on a scan attribute, memory configured to store predetermined first and second values for the scan attribute to define different first and second scan modes, respectively, a sensor configured to sense a posture state of a patient, and a controller configured to load the RF module with one of the predetermined first and second values for the scan attribute, based on the posture state determined, to cause the RF module to switch between the first and second scan modes.

Certain embodiments provide a system that includes a home-based patient care system (PCS) and an IMD. The PCS is configured to monitor at least one physiological attribute of a patient. The IMD is configured to wirelessly communicate with the PCS. The IMD may include an RF device configured to operate in first and second scan modes, a sensor configured to sense a posture state of a patient, and a controller configured to cause the RF module to switch between the first and second scan modes based on the posture state of the patient. In one embodiment, the controller causes the RF module to operate in the first scan mode when the posture state of the patient is vertical, and wherein the controller causes the RF module to operate in the second scan mode when the posture state of the patient is horizontal.

### BRIEF DESCRIPTION OF THE DRAWINGS

FIG. 1 is a simplified view of an exemplary implantable medical device (IMD) in electrical communication with at least three leads implanted into a patient's heart in accordance with an embodiment.

FIG. 2 is a simplified view of an IMD and patient care system (PCS) in accordance with an embodiment.

FIG. 3 illustrates a block diagram of an IMD in accordance with an embodiment.

FIG. 4 illustrates a flow chart of a process for calibrating a position sensor in accordance with an embodiment.

FIG. 5 illustrates a method of controlling RF scanning attributes of an IMD in accordance with an embodiment.

FIG. 6 illustrates a method of controlling RF scanning attributes of an IMD in accordance with an embodiment.

FIG. 7 illustrates a distributed processing system in accordance with an embodiment.

FIG. 8 illustrates a functional block diagram of an external device that is operated in accordance with the processes described herein and to interface with implantable medical devices as described herein.

FIG. 9 illustrates a block diagram of exemplary internal components of an IMD.

### DETAILED DESCRIPTION

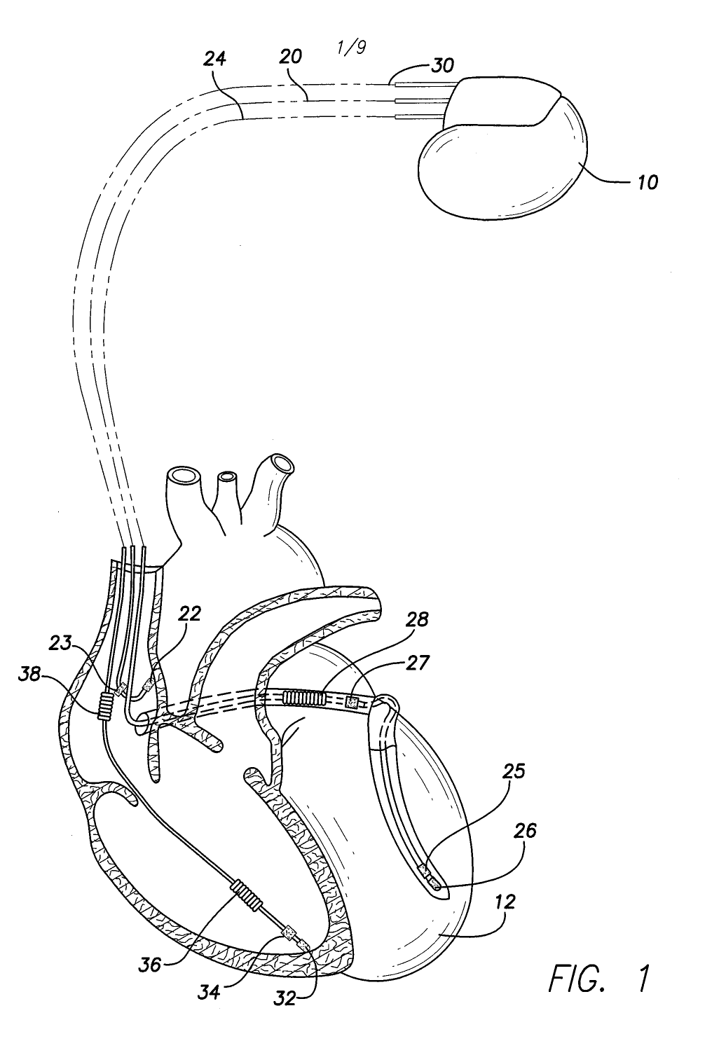

FIG. 1 illustrates an IMD 10 in electrical communication with a patient's heart 12 by way of three leads 20, 24 and 30 suitable for delivering multi-chamber stimulation and/or shock therapy. To sense atrial cardiac signals and to provide right atrial chamber stimulation therapy, the IMD 10 is coupled to an implantable right atrial lead 20 including at least one atrial tip electrode 22 that typically is implanted in the patient's right atrial appendage. The right atrial lead 20 may also include an atrial ring electrode 23 to allow bipolar stimulation or sensing in combination with the atrial tip electrode 22.

To sense the left atrial and left ventricular cardiac signals and to provide left-chamber stimulation therapy, the IMD 10 is coupled to a lead 24 designed for placement in the “coronary sinus region” via the coronary sinus ostium in order to place a distal electrode adjacent to the left ventricle and additional electrode(s) adjacent to the left atrium. As used herein, the phrase “coronary sinus region” refers to the venous vasculature of the left ventricle, including any portion of the coronary sinus, great cardiac vein, left marginal vein, left posterior ventricular vein, middle cardiac vein, and/or small cardiac vein or any other cardiac vein accessible by the coronary sinus.

Accordingly, the lead 24 is designed to: receive atrial and/or ventricular cardiac signals; deliver left ventricular pacing therapy using at least one left ventricular tip electrode 26 for unipolar configurations or in combination with left ventricular ring electrode 25 for bipolar configurations; deliver left atrial pacing therapy using at least one left atrial ring electrode 27 as well as shocking therapy using at least one left atrial coil electrode 28.

The IMD 10 is also shown in electrical communication with the patient's heart 12 by way of an implantable right ventricular lead 30 including, in the embodiment, a right ventricular (RV) tip electrode 32, a right ventricular ring electrode 34, a right ventricular coil electrode 36, a superior vena cava (SVC) coil electrode 38, and so on. Typically, the right ventricular lead 30 is inserted transvenously into the heart 12 so as to place the right ventricular tip electrode 32 in the right ventricular apex such that the RV coil electrode 36 is positioned in the right ventricle and the SVC coil electrode 38 will be positioned in the right atrium and/or superior vena cava. Accordingly, the right ventricular lead 30 is capable of receiving cardiac signals, and delivering stimulation in the form of pacing and shock therapy to the right ventricle.

The IMD may be one of various types of implantable devices, such as, for example, an implantable pacemaker, implantable cardioverter-defibrillator (“ICD”), neurostimulator, electrophysiology (“EP”) mapping and radio frequency (“RF”) ablation system, or the like.

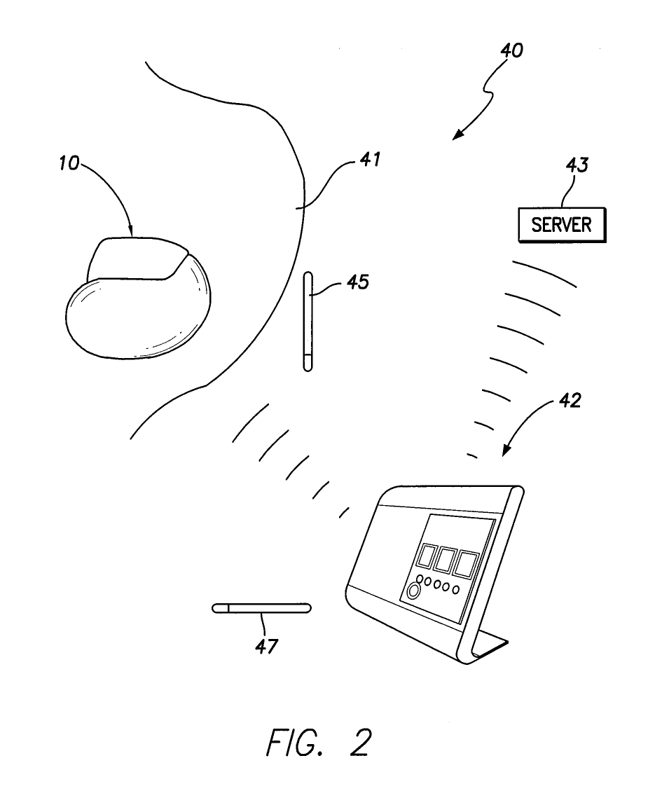

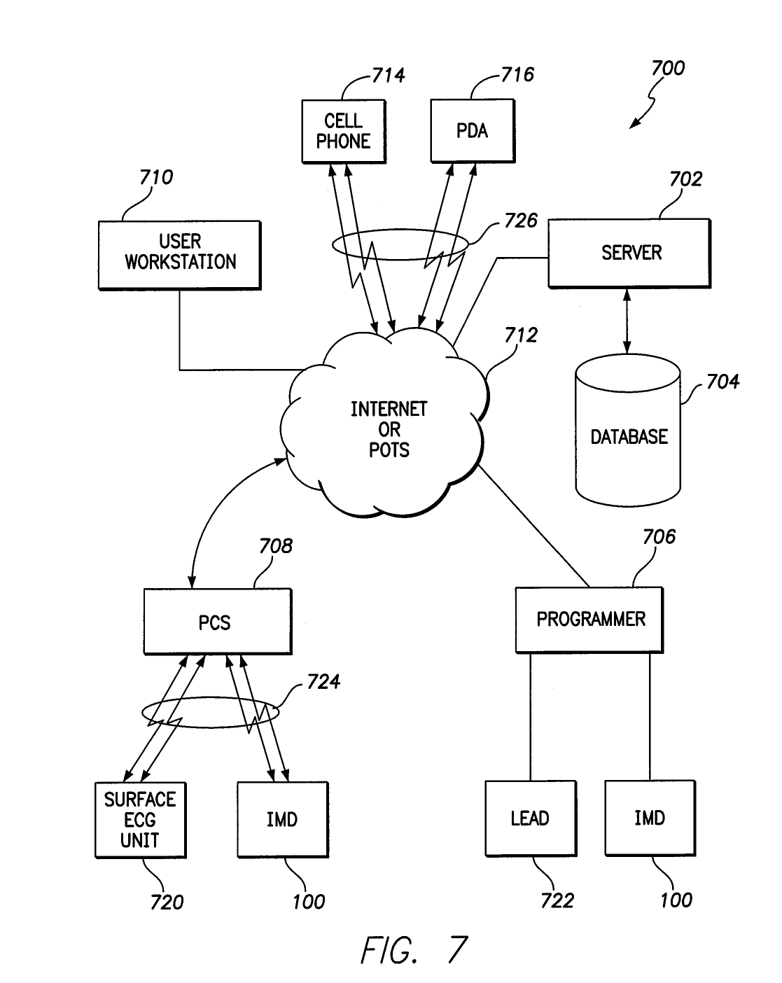

FIG. 2 is a simplified view of the IMD 10 and a patient care system (PCS) 42 in accordance with an embodiment. The IMD 10 is located within a patient 41. The remotely-located PCS 42 monitors the IMD 40. The PCS 42 may be located within the home of the patient 41, in their vehicle, at their office and the like. When, the PCS 42 is located within the patient's home, it may be proximate the patient's 41 bed. The PCS 42 functions as a base station that wirelessly communicates with the IMD 10. The PCS 42 also communications with a remote server 43 within a patient care network (as shown in FIG. 7) such as over a phone link, cellular link, Internet connection, local area network, wide area network and the like.

The PCS 42 performs various functions, such as operating as an intermediate relay device to collect and store patient physiologic data, IMD operational status data and the like. The PCS 42 then transmits the physiologic data, IMD operational status data and other data to the remote server 43 of the patient care network. Physicians and other personnel can monitor the patient and collect data over the patient care network. Also, the PCS 42 may receive updates, upgrades and other IMD control-related information from the patient care network and relay the IMD control-related information to the IMD 10.

However, the patient 41 is not always within communications range of the PCS 42 installed in their home. Thus, a home monitor session protocol is established as a manner by which the IMD 10 and the PCS 42 may initiate a communications session. In accordance with at least one embodiment, the home monitor session protocol provides that the IMD 10 shall have the ability to scan multiple different frequency bands and shall scan each frequency band utilizing a corresponding scan mode. The scan modes are defined by various scan attributes stored in the IMD 10. For example, a scan mode may be defined by the frequency band to be scanned, how often the frequency band is scanned (hereafter scan rate), the receive power level utilized by the IMD 10 to “listen” or scan the designated frequency band for connection requests, and the like. The home monitor session protocol may include 2 or more designated frequency bands, each of which is scanned with a different scan rate, scan receive power level, the channels to scan and the like. The scan rate defines one or more of the interval between scans, the length of each scan operation, particular times of day to initiate a scan operation and the like.

As one example, one scan mode may scan a first/high frequency range/band (e.g., 2.5 GHz band) at a first scan rate such as every minute, every 30 minutes, every hour, continuously and the like for connection requests. When a connection request is sensed in the first/high band, the IMD 10 initiates a communications session with the external device or base station requesting the connection, such as a programmer, the PCS 42 and the like. The communications session may be established over the first/high frequency band, a lower frequency band or any other frequency band that affords a more robust, reliable connection. The scan protocol may further provide that the IMD 10 shall scan a second/low frequency range/band (e.g., 400 MHz band) at another scan rate, such as at a predetermined periodic interval (e.g., every 2 hours, every 4 hours, during certain intervals of the day such as hourly between 8 pm and 8 am, every 2 hours between 9 pm and 6 am, and the like).

The first/high frequency band may be designated for “unscheduled” PCS or programmer follow up sessions, while the second/low frequency band may be designated for “scheduled” PCS sessions. One of the frequency bands may be designated as a primary frequency band, while the other frequency band is designated as a secondary frequency band. The secondary frequency band may be designated as the frequency band that only supports unidirectional communication (e.g. from the external device to the IMD), that affords a less robust or reliable connection link, is for unscheduled sessions or based on some other factor. The primary frequency band may be designated as the frequency band that supports bidirectional communications sessions, affords the most robust and reliable connection links, is designated for scheduled sessions or based on some other factors.

An unscheduled session may be initiated by the patent 41 or a physician at any time by selecting an “unscheduled IMD interrogation” mode or button on the PCS 42, on a programmer, and/or by using a wand 45. The wand 45 transmits a connection request over the high frequency band. If the IMD 10 is scanning the high frequency band in accordance with a home monitor session protocol, the IMD 10 will sense the connection request, and determine whether the connection request is intended for the IMD 10. For example, the IMD 10 may determine that a connection request is intended for it when the connection request includes a designated manufacturer ID and a device ID for the IMD 10. Each IMD 10 is loaded with a unique device ID. When the IMD 10 receives a connection request with its device ID, the IMD 10 identifies the connection request as valid and responds accordingly. If the connection request is intended for the IMD 10, the IMD 10 initiates a PCS 42 to IMD 10 communications session. The communications session may be initiated over the high frequency band, the low frequency band or any other frequency band designated for bidirectional communications.

A scheduled session is initiated automatically by the PCS 42 at scheduled intervals according to the home monitor scan session, such as in the middle of the night (while the patient sleeps), while commuting to work, over lunch while at work and the like. A wand 47, in the PCS is stationed at all times near the bed or a designated location where the patient is expected to be located during the scheduled intervals. For example, if the scheduled interval is during lunch, the PCS may be located at the patient's desk. If the scheduled interval is during morning or evening commuting time, the PCS may be positioned in the patient's car. If the scheduled interval is late at night, the PCS 42 is positioned near the patient's bed. At the predetermined times during the scheduled interval (e.g., throughout the night), the PCS 42 transmits connection requests over the selected frequency band.

The frequency ranges/bands are defined to afford different performance characteristics. For example, one band may afford reliable, robust long range connections, while another band may utilize less power to maintain, but experience a shorter range or less reliable connection. The low frequency band (e.g. 400 MHz, 900 MHz, etc.) affords a longer range and more robust connection than the high frequency band (e.g., 1.25 GHz, 2.54 GHz, etc.). However, the high frequency band draws less power from the IMD when scanning for connection requests and during a communications session.

As discussed above, however, there is no need for the IMD 10 to scan the high frequency band continuously in the middle of the night because it is very unlikely that the patient 41 will seek to initiate an unscheduled session (that is, the patient 41 will be asleep). Also, there is no need for the IMD 10 to scan the low frequency band, even periodically, during the daytime because it is very unlikely that the patient 41 will lay or sit next to the low frequency wand for an extended period of time to catch the next scheduled connection request from the PCS. The home monitor session protocol may take into account the behavior of the individual patient or the patient population in general when designating the patient posture states and times of day during which the IMD 10 will change scan modes. The protocol may also take into account the behavior of the individual patient or the patient population in general when designating the values for select scan attributes. These select scan attributes are then loaded into the IMD 10.

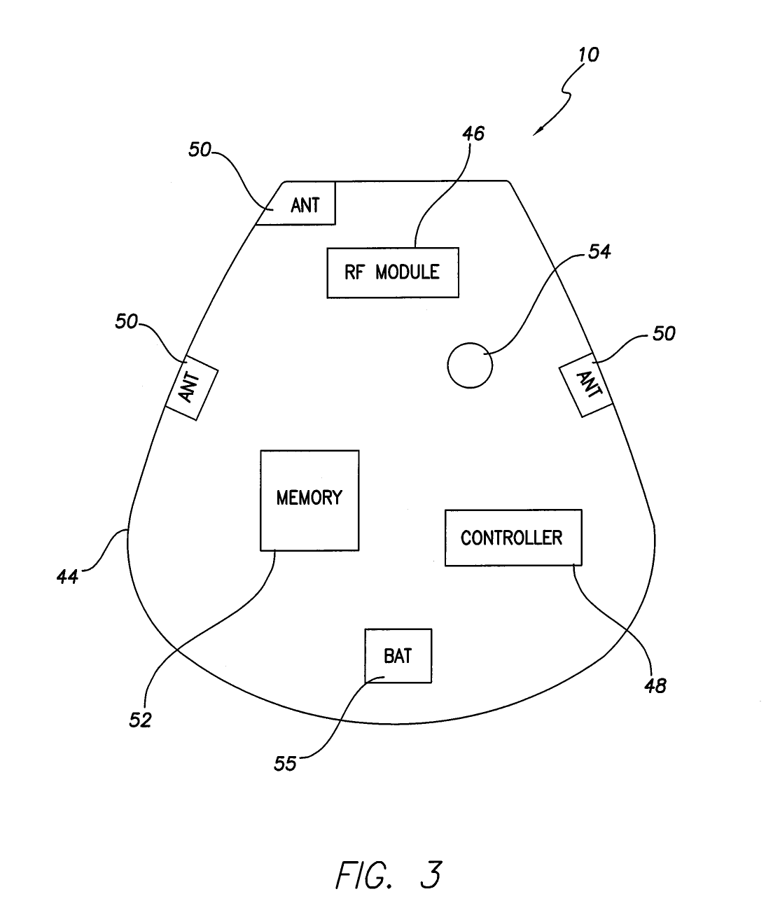

FIG. 3 illustrates a block diagram of the IMD 10. As shown in FIG. 3, the IMD 10 includes a main housing or body 44 that is configured to be implanted in the patient 41 (shown in FIG. 2). The IMD 10 contains an RF module 46, such as an RF chip, in electrical communication with a controller 48, memory 52 and an antenna 50. The controller 48 is also an electrical communication with memory 52 and a position sensor 54. The IMD 10 is powered through an internal battery 55.

The RF module 46 is configured to operate in multiple scan modes to search for connection requests from an external device and to establish an RF connection over a predetermined frequency band based on one or more scan attributes. The scan attributes for all available scan modes are loaded into memory 52. The RF module 46 is loaded with a set of scan attributes from the memory 52 to be configured to establish an RF connection over one or more of primary and/or secondary frequency bands that are non-overlapping and distinct from one another. The primary frequency range may be associated with one or more the scan modes, while the secondary frequency range may be associated with a different one or more scan modes. For example, first and second scan modes may designate different scan rates and scan powers to be used with the primary frequency band at different points in time or intervals. Different third and fourth scan modes may designate different scan rates and scan powers to be used with the secondary frequency band, at select points in time or select intervals.

In one embodiment, the scan attributes may represent scan rate. The RF module 46 may scan one or each of the predetermined frequency bands at different first and second rates when in the first and second scan modes, respectively. Optionally, the scan attributes may represent one or more of scan receive power, a frequency change of the predetermined frequency band, antenna impedance, antenna capacitance, antenna inductance, and the like.

The protocol may designate multiple channels to be used within a select frequency band. The channels may be separated through frequency shift key modulation, time division multiple access (TDMA), frequency division multiple access (FDMA), code division multiple access (CDMA) and the like. When in the first scan mode, for example, the RF module 46 may scan one or more channels within the predetermined frequency band for an RF connection request from an authorized external patient care system, such as the PCS 42 (shown in FIG. 2). For example, the first scan mode may include a scan for connection requests over multiple separate channels. The RF module 46 may operate at a first receive power level at which the RF module 46 is merely listening for a wake-up signal from the PCS 42. Once the RF module 46 receives this wake-up signal, the RF module 46 switches over to a second scan mode that supports bidirectional communication over a different frequency range. As noted, the first scan mode may be generally at low energy, while the second scan mode may be at a higher energy.

The second scan mode may scan a lower energy frequency range, but at an increased scan rate and increased receive power or gain. For example, the second scan mode may be at 400 MHz band. In this scan mode, the RF module 46 may transmit data to and receive data from the PCS 42. The RF module 46 may transmit physiological data, such as recorded cardiac events, and operational data of the IMD 10 stored in the memory 52 to the PCS 42. Once this data is transmitted from the RF module 46, optionally the data may then be removed from the memory 52 of the IMD 10, as that data is then stored for a longer time frame at the PCS 42 or network server. The data received at the PCS 42 may be used to adjust the settings of the IMD 10. In other words, the PCS 42 may use the received data to adapt the IMD 10 to compensate for changing physiological circumstances of the patient 41 and/or operations of the IMD 10.

The PCS 42 may also transmit updates, upgrades or other operating data back to the IMD 10. The operating data is then stored in the memory 52, and the controller 48 adjusts operation of the IMD 10 based on the updated operating data.

The memory 52 is loaded with sets of predetermined values for the scan attributes, such as a scan rate, to define the different scan modes, respectively, at the time of manufacture or at implant through a programmer. The controller 48 loads the RF module 46 with one set of the predetermined values for particular scan attributes. The values may change over time based on detected internal and external conditions. For example, the frequency ranges may be changed for different types of scan protocols and different types of communication sessions.

The position sensor 54 may be any type of position sensor, such as an inclinometer, level sensor, or the like. The position sensor 54 may include, for example, an asymmetrical body having one or more electromagnetic, optical, or the like emitters that are configured to be detected by separate receivers. In one example, the position sensor 54 may be shaped as an isosceles triangle with sensors or emitters at each vertex. However, any known position sensor that may be used to differentiate between vertical and horizontal orientations, for example, may be used.

By way of example, the position sensor 54 may be Three-dimensional Micro-Electro-Mechanical-Systems (3D MEMS) sensor. A 3D MEMS sensor can be fabricated in a tiny piece of silicon, capable of measuring acceleration in three orthogonal directions. Using the 3D MEMS sensor, the IMD 10 affords accurate inclination angle (e.g., within 1 arc minute) measurement, with mechanical damping for use in environments subject to strong vibration. The power requirements of the 3D MEMS sensors are extremely low, which gives them a significant advantage in battery-operated IMDs 10 (e.g., microampere average power consumption).

The position sensor 54 is used to sense the posture state of the patient 41. The sensor 54 may produce raw analog or digital signals representative of X-axis, Y-axis and Z-axis orientation of the patient relative to a coordinates system or reference item (e.g., direction of gravitational force, true magnetic North, etc.). Optionally, the sensor 54 may produce a resultant orientation measurement, such as pitch, yaw and roll angular orientations relative to reference coordinates or a reference item (e.g., direction of gravitational force). For example, based on calibrated X, Y, and Z axes, sensed by the position sensor 54, the controller 48 is able to determine the actual posture, such as vertical or horizontal, of the patient 41. The controller 48 may manually or automatically calibrate the IMD 10 to monitor for potential posture states including one or more of a supine state, prone state, right side position, or left side position.

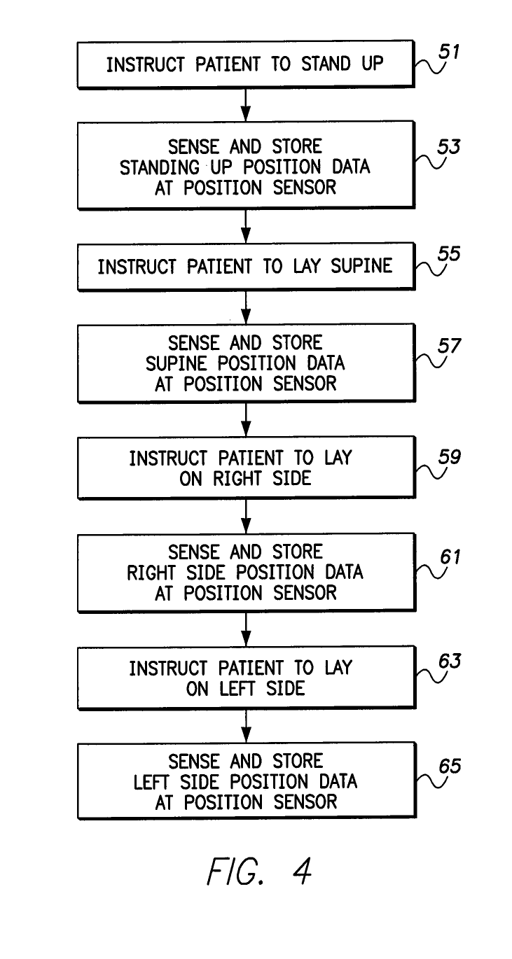

FIG. 4 illustrates a flow chart for a process to calibrate the position sensor 54 using an external programmer or the PCS 42, in accordance with an embodiment. At 51, the patient 41 having the implanted IMD 10, stands up and the external programmer or PCS 42 transmits a vertical state condition to the IMD 10 to inform the IMD 10 that the present readings by the sensor 54 correspond to the potential posture state of standing up or vertical. Next at 53, posture signals from the position sensor 54 are sensed for a test interval (e.g., 30 sec., 1 minute, 20 minutes). The posture signals for the current test interval are recorded as a calibrated potential posture state. For example, the potential posture state may constitute a single value or set of values representing integration of the posture signal over the test interval. Optionally, the potential posture state may represent a single posture signal or an average of multiple posture signals for the test intervals. At 53, the X, Y, and Z positions and/or orientations of the position sensor 54, while the patient 41 is standing, are also stored as vertical state reference coordinates.

At 55, the patient 41 is instructed to lay supine and the external programmer or PCS 42 transmits a horizontal state condition to the IMD 10 to inform the IMD 10 that the present readings by the sensor 54 correspond to the potential posture state of laying down or horizontal. At 57, the X, Y, and Z positions and/or orientations of the position sensor 54, while the patient 41 is lying supine, are sensed and stored as horizontal reference coordinates.

At 59, the patient 41 is instructed to lay on his/her right side and the external programmer or PCS 42 transmits a right side state condition to the IMD 10 to inform the IMD 10 that the present readings by the sensor 54 correspond to the potential posture state of laying on the right side. At 61, the X, Y, and Z positions and/or orientations of the position sensor 54, while the patient 41 is lying on his/her right side, are sensed and stored as right side reference coordinates. At 63, the patient 41 is instructed to lay on his/her left side and the external programmer transmits a left side state condition to the IMD 10 to inform the IMD 10 that the present readings by the sensor 54 correspond to potential posture states of laying on the left side. At 65, the X, Y, and Z positions and/or orientations of the position sensor 54, while the patient is lying on his/her left side, are sensed and stored as left side reference coordinates. In this manner, the position sensor 54 is calibrated with respect to upright, supine, and side-lying down positions relative to a reference coordinate system such as defined by gravity. For example, the horizontal axis may be perpendicular to the direction of gravity, while the vertical axis is parallel to the direction of gravity.

Referring again to FIG. 3, as the name suggests, the position sensor 54 detects a posture of a patient 41, in which the IMD 10 is implanted. As noted above, the position sensor 54, once calibrated, is used to discern potential posture states and to identify an actual posture state between upright, supine, and right side, left side positions/orientations.

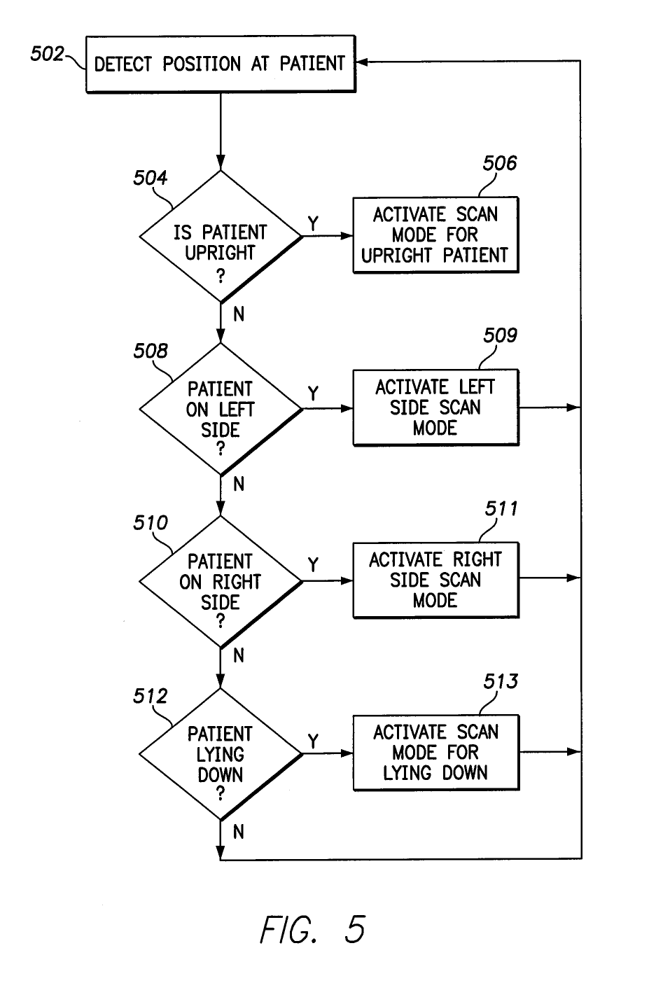

FIG. 5 illustrates a method of controlling RF scanning attributes of an IMD 10 in accordance with an embodiment. At 502, the controller 48, through the position sensor 54, receives sensed signals from the position sensor 54. Optionally, the position sensor 54 repeatedly outputs one or more posture signals to the controller 48. Alternatively, the controller 48 may periodically read the posture signals.

At 504, the controller 48 determines if the patient is upright. For example, if the position sensor 54 is configured such that X, Y, and Z axes are at a particular orientation (relative to gravity) the controller 48 determines that the patient 41 is upright when data, corresponding to this orientation, is output from the position sensor 54. For example, a posture signal from sensor 54 may be compared to potential posture states created during calibration. The potential posture state that closest resembles or matches the posture signal is declared to be the actual posture state. As one example, the posture signal may be integrated over a time interval to form a posture signal sum that is then compared to thresholds that represent the potential posture states. As another example, the potential posture states may represent pre-recorded calibration signals from the sensor 54 recorded over time during calibration. A new real-time current posture signal from the position sensor 54 may be collected for a state test interval (e.g., 1 minute, 20 minutes, 30 seconds). The current posture signal is compared to the calibration signals for the potential posture states. The comparison may be subtraction, correlation and the like. When the actual posture state is determined to be upright or vertical, flow moves to 506.

At 506, the controller 48 identifies the scan mode that has been designated for operation when the patient is in the upright or vertical orientation. The controller 48 loads the RF module 46 with one value or a set of values for the scan attribute(s), associated with the scan mode that corresponds to the posture state determined, to cause the RF module 46 to switch to this new scan mode. For example, the controller 48 may load a desired set of values for scan attributes into certain registers in the RF module 46. If the controller 48 determines that the position sensor 54 detects that the patient 41 is upright, as one example, the controller 48 may instruct the RF module 46 to scan for communication over a 2.45 GHz band at a set receive power level, at a set scan rate, and for set scan intervals.

Returning to 504, if the data signal from the position sensor 54 does not correspond to an upright position, then flow moves to 508.

At 508, the controller 48 determines whether the position sensor 54 detects that the patient 41 is laying horizontally but on his/her left side. If so, flow moves to 509 where the controller 48 may instruct the RF module 46 to scan for communication over a 400 MHz band at a different receiver power, scan rate and for a different scan interval. As another example, when on his/her left side, the RF module 46 may be connected to a different antenna (e.g. antenna having different orientations and/or positions within the IMD 10). As a further example, the RF module 46 may be tuned with different antenna impedance, capacitance and inductance based on whether the patient is laying on his/her left side or right side. If the data signal from the position sensor 54 does not correspond to a left side orientation, then flow moves to 510.

At 510, the controller 48 determines whether the position sensor 54 detects that the patient 41 is laying horizontally but on his/her right side. If so, then flow moves to 511 where the controller 48 may instruct the RF module 46 to to scan for communication over a select frequency band, ii) to connect to a different antenna, iii) to tune with different antenna impedance, capacitance and inductance, and the like. If the signal data from the position sensor 54 does not correspond to a right side orientation, then flow moves to 512.

At 512, the controller 48 determines whether the position sensor 54 detects that the patient 41 is supine 41. If so, then flow moves to 513 where the controller 48 may instruct the RF module 46 to scan for communication over a 400 MHz band at a different receiver power, scan rate and for a different scan interval. Optionally, the controller 48 may instruct the RF module 46 i) to scan for communication over a select frequency band, ii) to connect to a different antenna, iii) to tune with different antenna impedance, capacitance and inductance, and the like. If the data from the position sensor 54 does not correspond to a supine orientation, then flow moves returns to 502.

Again, in the horizontal position, the patient is likely laying down in his/her bed by the PCS 42, and thus, the PCS 42 is configured to enter the 400 MHz data transfer scan mode during sleeping hours.

The PCS 42 may transmit scheduled data transfers at certain, predetermined times. The scheduled data transfer ideally occurs when the patient 41 is asleep, in close proximity to the PCS 42. During this time, the controller 48 may deactivate a 2.45 GHz scan mode, and instead simply passively await a 400 MHz scan mode, in which data is transferred between the PCS 42 and the IMD 10. Therefore, when the controller 48 determines by way of the position sensor 54 that the patient 41 is supine or otherwise horizontal, the controller 48 may deactivate the 2.45 GHz scan mode, and activate the passive 400 MHz scan mode.

Conversely, when the controller 48 determines through the position sensor 54 that the patient 41 is upright, the controller 48 switches to the 2.45 scan mode and deactivates the 400 MHz scan mode. In the 2.45 GHz mode, the RF module 46 may listen for a data transfer request from the base station over a predetermined frequency or frequency range. If the PCS 42 transmits a data transfer request over the predetermined frequency, the RF module 46 receives the request through the antenna 50, and communicates the request to the controller 48. The controller 48 then switches between the first scan mode at 2.45 GHz to the second scan mode at 400 MHz to receive data from the PCS 42 and transmit data from the memory 52 to the PCS 42 by way of the RF module 46. Moreover, when the controller 48 determines that the patient is lying down, through the position sensor 54, the controller 48 deactivates the 2.45 GHz scan mode, and, instead, enters a passive 400 MHz scan mode.

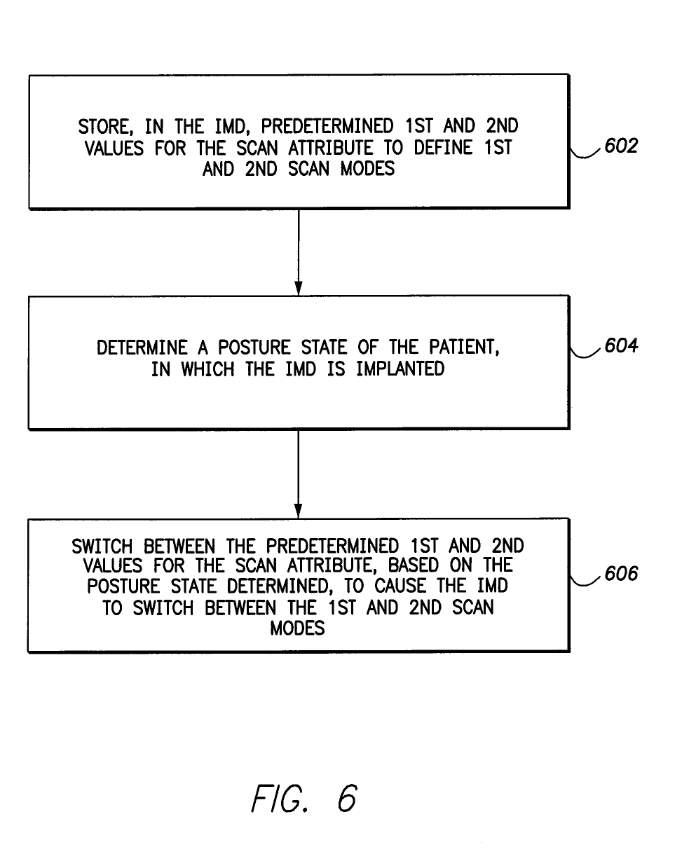

FIG. 6 illustrates a method of controlling RF scanning attributes of an IMD in accordance with an embodiment. At 602, predetermined first and second values for one or more scan attributes are stored in the IMD at the time of manufacture or through a programmer at calibration or at implant, to define first and second scan modes. At 604, a posture state of a patient is determined. For example, through the position sensor, it is determined whether the patient is upright or laying down. At 606, the IMD is switched between the predetermined first and second values for the scan attribute, based on the posture state determined, to cause the IMD to switch between the first and second scan modes.

Thus, embodiments provide an IMD having an RF-equipped chip or module that scans for possible RF connections more or less frequently during certain times based on patient positions. Consequently, the remote PCS unit may connect to the IMD in more frequent and targeted intervals while reducing the IMD's battery consumption.

FIG. 7 illustrates a distributed processing system 700 in accordance with one embodiment. The distributed processing system 700 includes a server 702 connected to a database 704, a programmer 706, a PCS 708 and a user workstation 710 electrically connected to a communication system 712. The communication system 712 may be the internet, a voice over IP (VolP) gateway, a local plain old telephone service (POTS) such as a public switched telephone network (PSTN), a cellular phone based network, and the like. Alternatively, the communication system 712 may be a local area network (LAN), a campus area network (CAN), a metropolitan area network (MAN), or a wide area network (WAM). The communication system 712 serves to provide a network that facilitates the transfer/receipt of information such as cardiac signal waveforms, ventricular and atrial heart rates and the like.

The server 702 is a computer system that provides services to other computing systems over a computer network. The server 702 controls the communication of information such as cardiac signal waveforms, ventricular and atrial heart rates, and detection thresholds. The server 702 interfaces with the communication system 712 to transfer information between the programmer 706, the local PCS 708, the user workstation 710 as well as a cell phone 714 and a personal data assistant (PDA) 716 to the database 704 for storage/retrieval of records of information. On the other hand, the server 702 may upload raw cardiac signals from an implanted lead 722, surface ECG unit 722 or the IMD 100 via the local PCS 708 or the programmer 706.

The database 704 stores information such as cardiac signal waveforms, ventricular and atrial heart rates, detection thresholds and the like, for a single or multiple patients. The information is downloaded into the database 704 via the server 702 or, alternatively, the information is uploaded to the server from the database 704. The programmer 706 is similar to the external device 600 and may reside in a patient's home, a hospital, or a physician's office. The programmer 706 interfaces with the lead 722 and the IMD 100. The programmer 706 may wirelessly communicate with the IMD 100 and utilize protocols, such as Bluetooth, GSM, infrared wireless LANs, HIPERLAN, 3G, satellite, as well as circuit and packet data protocols, and the like. Alternatively, a hard-wired connection may be used to connect the programmer 706 to the IMD 100. The programmer 706 is able to acquire cardiac signals from the surface of a person (e.g., ECGs), intra-cardiac electrogram (e.g., IEGM) signals from the IMD 700, and/or cardiac signal waveforms, ventricular and atrial heart rates, and detection thresholds from the IMD 100. The programmer 706 interfaces with the communication system 712, either via the internet or via POTS, to upload the information acquired from the surface ECG unit 720, the lead 722 or the IMD 100 to the server 702.

The local PCS 708 interfaces with the communication system 712 to upload one or more of cardiac signal waveforms, ventricular and atrial heart rates, and detection thresholds to the server 702. In one embodiment, the surface ECG unit 720 and the IMD 700 have a bi-directional connection 724 with the local PCS 708 via a wireless connection. The local PCS 708 is able to acquire cardiac signals from the surface of a person, intra-cardiac electrogram signals from the IMD 100, and/or cardiac signal waveforms, ventricular and atrial heart rates, and detection thresholds from the IMD 100. On the other hand, the local PCS 708 may download stored cardiac signal waveforms, ventricular and atrial heart rates, and detection thresholds and the like, from the database 704 to the surface ECG unit 720 or the IMD 100.

The user workstation 710 may interface with the communication system 712 via the internet or POTS to download cardiac signal waveforms, ventricular and atrial heart rates, and detection thresholds via the server 702 from the database 704. Alternatively, the user workstation 710 may download raw data from the surface ECG units 720, lead 722 or IMD 700 via either the programmer 706 or the local PCS 708. Once the user workstation 710 has downloaded the cardiac signal waveforms, ventricular and atrial heart rates, or detection thresholds, the user workstation 710 may process the information in accordance with one or more of the operations described above. The user workstation 710 may download the information and notifications to the cell phone 714, the PDA 716, the local PCS 708, the programmer 706, or to the server 702 to be stored on the database 704. For example, the user workstation 710 may communicate data to the cell phone 714 or PDA 716 via a wireless communication link 726.

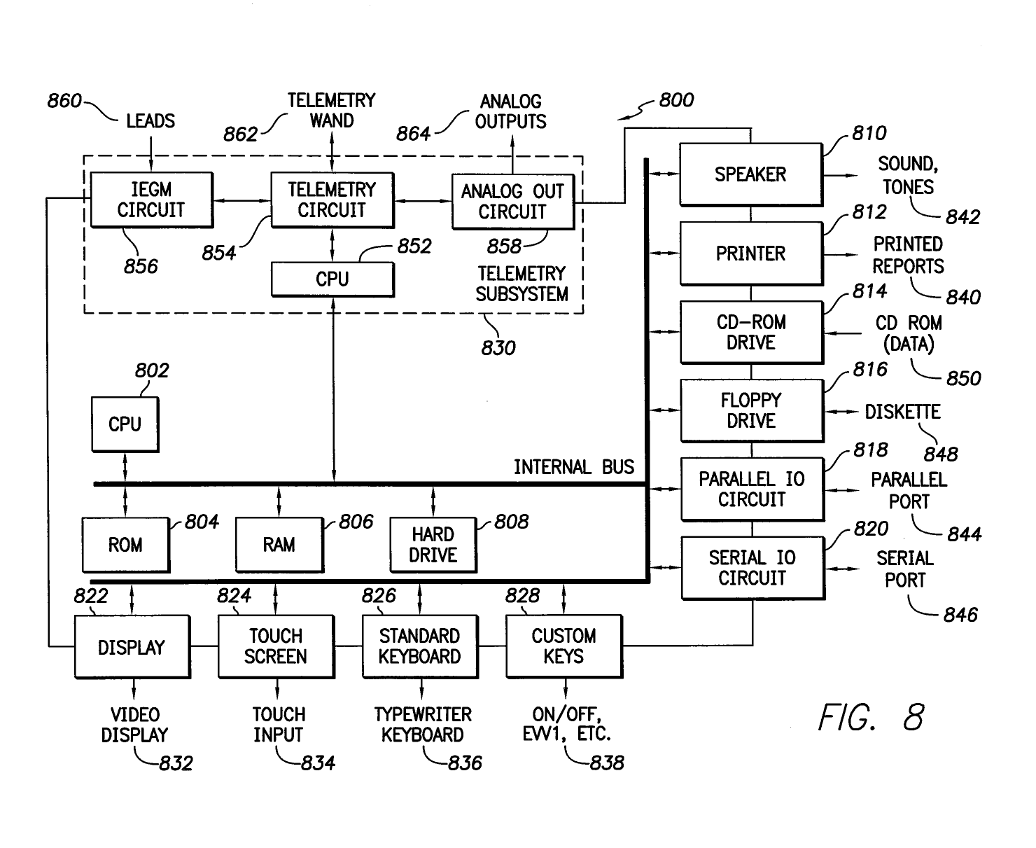

FIG. 8 illustrates a functional block diagram of an external device 800 (e.g., a PCS or programmer) that is operated in accordance with the processes described herein and to interface with implantable medical devices as described herein. The external device 800 (PCS or programmer) may be a workstation, a portable computer, an IMD programmer, a PDA, a cell phone and the like. The external device 800 includes an internal bus that connects/interfaces with a Central Processing Unit (CPU) 802, ROM 804, RAM 806, a hard drive 808, the speaker 810, a printer 812, a CD-ROM drive 814, a floppy drive 816, a parallel I/O circuit 818, a serial I/O circuit 820, the display 822, a touch screen 824, a standard keyboard connection 826, custom keys 828, and a telemetry subsystem 830. The internal bus is an address/data bus that transfers information between the various components described herein. The hard drive 808 may store operational programs as well as data, such as waveform templates and detection thresholds.

The CPU 802 typically includes a microprocessor, a micro-controller, or equivalent control circuitry, designed specifically to control interfacing with the external device 800 and with the IMD 10. The CPU 802 performs the COI measurement process discussed above. The CPU 802 may include RAM or ROM memory, logic and timing circuitry, state machine circuitry, and I/O circuitry to interface with the IMD 10. The display 822 (e.g., may be connected to the video display 832). The touch screen 824 may display graphic information relating to the IMD 10. The display 822 displays various information related to the processes described herein. The touch screen 824 accepts a user's touch input 834 when selections are made. The keyboard 826 (e.g., a typewriter keyboard 836) allows the user to enter data to the displayed fields, as well as interface with the telemetry subsystem 830. Furthermore, custom keys 828 turn on/off 838 (e.g., EVVI) the external device 800. The printer 812 prints copies of reports 840 for a physician to review or to be placed in a patient file, and speaker 810 provides an audible warning (e.g., sounds and tones 842) to the user. The parallel I/O circuit 818 interfaces with a parallel port 844. The serial I/O circuit 820 interfaces with a serial port 846. The floppy drive 816 accepts diskettes 848. Optionally, the floppy drive 816 may include a USB port or other interface capable of communicating with a USB device such as a memory stick. The CD-ROM drive 814 accepts CD ROMs 850.

The telemetry subsystem 830 includes a central processing unit (CPU) 852 in electrical communication with a telemetry circuit 854, which communicates with both an IEGM circuit 856 and an analog out circuit 858. The circuit 856 may be connected to leads 860. The circuit 856 is also connected to the implantable leads 114, 116 and 118 to receive and process IEGM cardiac signals as discussed above. Optionally, the IEGM cardiac signals sensed by the leads 114, 116 and 118 may be collected by the IMD 10 and then transmitted, to the external device 800, wirelessly to the telemetry subsystem 830 input.

The telemetry circuit 854 is connected to a telemetry wand 862. The analog out circuit 858 includes communication circuits to communicate with analog outputs 864. The external device 800 may wirelessly communicate with the IMD 100 and utilize protocols, such as Bluetooth, GSM, infrared wireless LANs, HIPERLAN, 3G, satellite, as well as circuit and packet data protocols, and the like. Alternatively, a hard-wired connection may be used to connect the external device 800 to the IMD 10.

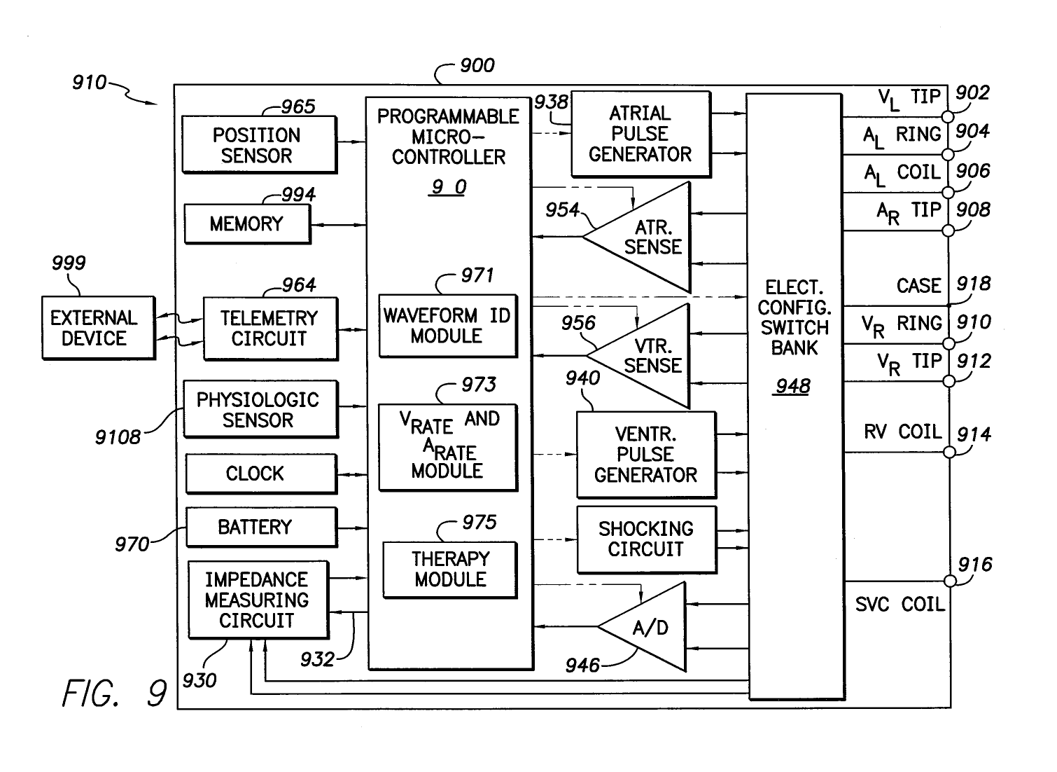

FIG. 9 illustrates a block diagram of exemplary internal components of an IMD 910. The IMD 910 is for illustration purposes only, and it is understood that the circuitry could be duplicated, eliminated or disabled in any desired combination to provide a device capable of treating the appropriate chamber(s) with cardioversion, defibrillation and/or pacing stimulation as well as providing for apnea detection and therapy. The housing 900 for IMD 910, shown schematically in FIG. 9, is often referred to as the “can”, “case” or “case electrode” and may be programmably selected to act as the return electrode for all “unipolar” modes. The housing 900 may further be used as a return electrode alone or in combination with one or more of the coil electrodes for shocking purposes. The housing 900 further includes a connector (not shown) having a plurality of terminals(shown schematically and, for convenience, the names of the electrodes to which they are connected are shown next to the terminals). A right atrial tip terminal (A.sub.R TIP) 908 is adapted for connection to the atrial tip electrode. A left ventricular tip terminal (V.sub.L TIP) 902, a left atrial ring terminal (A.sub.L RING) 904, and a left atrial shocking terminal (A.sub.L COIL) 906 are adapted for connection to the left ventricular ring electrode, the left atrial tip electrode, and the left atrial coil electrode, respectively. A right ventricular tip terminal (V.sub.R TIP) 912, a right ventricular ring terminal (V.sub.R RING) 910, a right ventricular shocking terminal (R.sub.V COIL) 914, and an SVC shocking terminal (SVC COIL) 916 are adapted for connection to the right ventricular tip electrode, right ventricular ring electrode, the RV coil electrode, and the SVC coil electrode, respectively.

The IMD 910 includes a programmable microcontroller 920 which controls operation. The microcontroller 920 (also referred to herein as a processor module or unit) typically includes a microprocessor, or equivalent control circuitry, designed specifically for controlling the delivery of stimulation therapy and may further include RAM or ROM memory, logic and timing circuitry, state machine circuitry, and I/O circuitry. Typically, the microcontroller 920 includes the ability to process or monitor input signals (data) as controlled by program code stored in memory. The details of the design and operation of the microcontroller 920 are not critical to the invention. Rather, any suitable microcontroller 920 may be used that carries out the functions described herein. Among other things, the microcontroller 920 receives, processes, and manages storage of digitized cardiac data sets from the various sensors and electrodes. For example, the cardiac data sets may include IEGM data, pressure data, heart sound data, and the like.

The IMD 910 includes an atrial pulse generator 938 and a ventricular pulse generator 940 to generate pacing stimulation pulses for delivery by the right atrial lead, the right ventricular lead, and/or the coronary sinus lead via an electrode configuration switch 948. It is understood that in order to provide stimulation therapy in each of the four chambers of the heart, the atrial and ventricular pulse generators, 938 and 940, may include dedicated, independent pulse generators, multiplexed pulse generators or shared pulse generators. The pulse generators, 938 and 940, are controlled by the microcontroller 920 via appropriate control signals, and to trigger or inhibit the stimulation pulses.

The microcontroller 920 further includes timing control circuitry used to control the timing of such stimulation pulses (e.g., pacing rate, atria-ventricular (AV) delay, atrial interconduction (A-A) delay, or ventricular interconduction (V-V) delay, etc.) as well as to keep track of the timing of refractory periods, blanking intervals, noise detection windows, evoked response windows, alert intervals, marker channel timing, and the like. Switch 948 includes a plurality of switches for connecting the desired electrodes to the appropriate I/O circuits, thereby providing complete electrode programmability. Accordingly, the switch 948, in response to a control signal from the microcontroller 920, determines the polarity of the stimulation pulses (e.g., unipolar, bipolar, etc.) by selectively closing the appropriate combination of switches (not shown) as is known in the art.

Atrial sensing circuit 954 and ventricular sensing circuit 956 may also be selectively coupled to the right atrial lead, coronary sinus lead, and the right ventricular lead, through the switch 948 for detecting the presence of cardiac activity in each of the four chambers of the heart. Accordingly, the atrial (ATR SENSE) and ventricular (VTR SENSE) sensing circuits, 954 and 956, may include dedicated sense amplifiers, multiplexed amplifiers or shared amplifiers. The outputs of the atrial and ventricular sensing circuits, 954 and 956, are connected to the microcontroller 920 which, in turn, are able to trigger or inhibit the atrial and ventricular pulse generators, 938 and 940, respectively, in a demand fashion in response to the absence or presence of cardiac activity in the appropriate chambers of the heart.

Cardiac signals are also applied to the inputs of an analog-to-digital (A/D) data acquisition system 946. The data acquisition system 946 is configured to acquire IEGM signals, convert the raw analog data into a digital IEGM signal, and store the digital IEGM signals in memory 994 for later processing and/or telemetric transmission to an external device 999. The data acquisition system 946 is coupled to various leads through the switch 974 to sample cardiac signals across any combination of desired electrodes. The data acquisition system 946 is also coupled, through switch 974, to one or more of the sensors. The data acquisition system 946 acquires, performs ND conversion, produces and saves the digital pressure data, and/or acoustic data.

The controller 920 includes an analysis module 971, a therapy module 975, and a setting module 973 that function in accordance with embodiments described herein. The analysis module 971 analyzes a characteristic of interest from the heart. The setting module 973 sets a desired value for the pacing parameter based on the characteristic of interest from the heart. The pacing parameter may represent at least one of an AV delay, a VV delay, a VA delay, intra-ventricular delays, electrode configurations and the like. The controller 920 changes at least one of the AV delay, the VV delay, the VA delay, the intra-ventricular delays, electrode configurations and like in order to reduce systolic turbulence and regurgitation. The therapy module 973 determines what therapy to apply.

By way of example, the controller 920 may utilize different combinations of the electrodes on the lead (as the change in pacing parameters) to deliver different pacing stimulus when analyzing the characteristic of interest in the heart. As another example, the controller 920 may utilize different timing configurations associated with left ventricular sensing (as the change in pacing parameters) when analyzing the characteristic of interest. The analysis module 971 may analyze various signals from a variety of sensors to detect arrhythmias, and other characteristic of interest. The setting module 973 may set desired values for various operational parameters and physiologic parameters.

The microcontroller 920 is coupled to memory 994 by a suitable data/address bus, wherein the programmable operating parameters used by the microcontroller 920 are stored and modified, as required, in order to customize the operation of IMD 910 to suit the needs of a particular patient. The memory 994 also stores data sets (raw data, summary data, histograms, etc.), such as the IEGM data, heart sound data, pressure data, Sv02 data and the like for a desired period of time (e.g., 1 hour, 24 hours, 1 month). The memory 994 may store instructions to direct the microcontroller 920 to analyze the cardiac signals and heart sounds identify characteristics of interest and derive values for predetermined statistical parameters. The IEGM, pressure, and heart sound data stored in memory 994 may be selectively stored at certain time intervals, such as 5 minutes to 1 hour periodically or surrounding a particular type of arrhythmia of other irregularity in the heart cycle. For example, the memory 994 may store data for multiple non-consecutive 10 minute intervals.

The pacing and other operating parameters of the IMD 910 may be non-invasively programmed into the memory 994 through a telemetry circuit 964 in telemetric communication with the external device 999, such as a programmer, trans-telephonic transceiver or a diagnostic system analyzer, or with a remote monitor. The telemetry circuit 964 operates in various scan modes the manner discussed herein based on scan attributes loaded from the controller 920. The telemetry circuit 964 communicates with the microcontroller 920 through control signals. The telemetry circuit 964 scans for connection requests, establishes a communications link or session and allows data transfer to/from the external device 999 such as a base station PSC. The transferred data may include one or more of intra-cardiac electrograms, pressure data, acoustic data, Sv02 data, and status information relating to the operation of IMD 910 (as contained in the microcontroller 920 or memory 994) to be sent to the external device 999 through an established communication link.

The memory 994 may be programmed with multiple conditions that, when satisfied by the indicators, are representative of potential ischemic episodes. For example, the conditions may include one or more of i) amplitudes and/or durations for heart sounds S1, S2, S3 and/or S4, ii) timing, intervals between and/or deviation of events of interest (e.g., mitral valve closing, mitral valve opening, aortic valve closing, aortic valve opening), iii) amplitudes and durations of points in the IEGM signal, and iv) durations of systolic interval, diastolic interval, isovolumic relaxation interval, and/or isovolumic contraction interval. The conditions may be preprogrammed from an external device or automatically set by the IMD 910 based on prior operation and historic data collected from the patient.

The IMD 910 includes a position sensor 965 which operates as discussed herein to generate posture signals that are used by the controller 920 to identify an actual posture state of the patient. The actual posture state is then used by the controller 920 to determine the set of scan attributes to be loaded into the telemetry circuit 964.

The IMD 910 includes an accelerometer or other physiologic sensor 9108, commonly referred to as a “rate-responsive” sensor because it is typically used to adjust pacing stimulation rate according to the exercise state of the patient. Optionally, the physiological sensor 9108 may further be used to detect changes in cardiac output, changes in the physiological condition of the heart, or changes in activity (e.g., detecting sleep and wake states) and to detect arousal from sleep. While shown as being included within IMD 910, it is to be understood that the physiologic sensor 9108 may also be external to IMD 910, yet still be implanted within or carried by the patient. A common type of rate responsive sensor is an activity sensor incorporating an accelerometer or a piezoelectric crystal, which is mounted within the housing 900 of IMD 910.

The physiologic sensor 9108 may be used as an acoustic sensor that is configured to detect the heart sounds. For example, the physiologic sensor 9108 may be an accelerometer that is operated to detect acoustic waves produced by blood turbulence and vibration of the cardiac structures within the heart (e.g., valve movement, contraction and relaxation of chamber walls and the like). When the physiologic sensor 9108 operates as an acoustic sensor, it may supplement or replace other acoustic sensors. Other types of physiologic sensors are also known, for example, sensors that sense the oxygen content of blood, respiration rate and/or minute ventilation, pH of blood, ventricular gradient, etc. However, any sensor may be used which is capable of sensing a physiological parameter that corresponds to the exercise state of the patient and, in particular, is capable of detecting arousal from sleep or other movement.

The IMD 910 additionally includes a battery 970, which provides operating power to all of the circuits shown. The IMD 910 is shown as having impedance measuring circuit 930 which is enabled by the microcontroller 920 via a control signal 932. Herein, impedance is primarily detected for use in evaluating ventricular end diastolic volume (EDV) but is also used to track respiration cycles. Other uses for an impedance measuring circuit include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring stroke volume; and detecting the opening of heart valves, etc. The impedance measuring circuit 930 is advantageously coupled to the switch 948 so that impedance at any desired electrode may be obtained.

In contrast to a conventional device, embodiments provide a system and method in which the external, remote PCS or base station initiates communication with the IMD. The IMD, through the RF module, listens for the external communication in a first, low power scan mode (such as 2.45 GHz at less than 1 mA) at a first frequency. Once the IMD determines that the base station has initiated communication, the RF module of the IMD switches to the second scan mode, which may be a higher power mode configured to exchange data between the base station and the IMD (such as 400 MHz at 4 mA).

Thus, embodiments provide an IMD having an RF-equipped chip or module that scans for possible RF connections more or less frequently during certain times based on patient positions. Consequently, the remote base unit may connect to the IMD in more frequent and targeted intervals while reducing the IMD's battery consumption.

Therefore, embodiments provide a system and method that conserves battery power of the IMD. Optionally, other frequency bands may be used.

Optionally, the methods and systems described herein may be implemented in connection with an implanted or external neurostimulation device such as, but without limitations, the devices described in U.S. Pat. Nos. 7,983,762; 7,738,963; 7,684,866; and 7,532,936, all of which are incorporated by reference herein in their entireties.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, types of materials and coatings described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112, sixth paragraph, unless and until such claim limitations expressly use the phrase “means for” followed by a statement of function void of further structure.

What is claimed is:

1. A method for controlling radio frequency (RF) scanning attributes of an implantable medical device (IMD), comprising:
configuring an IMD to establish an RF connection with an external device over a predetermined frequency band based on at least one scan attribute;
storing, in the IMD, predetermined first and second values for the scan attribute to define different first and second scan modes, respectively;
determining a posture state of a patient, in which the IMD is implanted; and
switching between the predetermined first and second values for the scan attribute, based on the posture state determined, to cause the IMD to switch between the first and second scan modes.

2. The method of claim 1, wherein the determining includes sensing a posture of a patient from a sensor within the IMD, the sensor providing a posture signal and comparing the posture signal to potential posture states to identify an actual posture state.

3. The method of claim 1, wherein the configuring further comprises configuring the IMD to establish RF connections over primary and secondary frequency bands that are non-overlapping and distinct from one another.

4. The method of claim 1, wherein the scan attribute represents scan rate, the method further comprising scanning the predetermined frequency band at different first and second scan rates when in the first and second scan modes, respectively.

5. The method of claim 1, wherein the scan attribute represents at least one of scan rate, scan receive power, a frequency range of the predetermined frequency band, antenna impedance, antenna capacitance, and antenna inductance.

6. The method of claim 1, further comprising calibrating the IMD to monitor for potential posture states including at least one of a supine state, prone state, right side position and left side position.

7. The method of claim 1, further comprising, when in the first scan mode, scanning channels in the predetermined frequency band for an RF connection request from an authorized external patient care system.

8. The method of claim 1, further comprising, when in the first scan mode, scanning the predetermined frequency band at a first scan rate for an RF connection request and, when in the second scan mode, scanning the predetermined frequency band at a second scan rate for the RF connection request.

9. The method of claim 1, further comprising, in response to receiving an RF connection request over the predetermined frequency band, initiating scanning of a secondary frequency band for communications data from an external device.

10. An implantable medical device (IMD), comprising:
an RF module configured to operate in first and second scan modes to establish an RF connection over a predetermined frequency band based on a scan attribute; memory configured to store first and second values for the scan attribute to define different first and second scan modes, respectively;
a sensor configured to sense a posture state of a patient; and
a controller configured to load the RF module with one of the first and second values for the scan attribute, based on the posture state determined, to cause the RF module to switch between the first and second scan modes.

11. The IMD of claim 10, wherein the sensor senses a posture of a patient, in which the IMD is implanted, the sensor outputting a posture signal, the controller comparing the posture signal to potential posture states to identify an actual posture state of the patient.

12. The IMD of claim 10, wherein the RF module is configured to establish RF connections over primary and secondary frequency bands that are non-overlapping and distinct from one another.

13. The IMD of claim 10, wherein the scan attribute represents scan rate, the RF module scanning the predetermined frequency band at different first and second rates when in the first and second scan modes, respectively.

14. The IMD of claim 10, wherein the scan attribute represents at least one of scan rate, scan receive power, a frequency range of the predetermined frequency band, antenna impedance, antenna capacitance, and antenna inductance.

15. The IMD of claim 10, wherein the controller is configured to calibrate the IMD to monitor for potential posture states including at least one of a supine state, prone state, right side position and left side position.

16. The IMD of claim 10, wherein the RF module, when in the first scan mode, scans channels in the predetermined frequency band for an RF connection request from an authorized external patient care system.

17. The IMD of claim 10, wherein the RF module, when in the first scan mode, scans the predetermined frequency band at a first scan rate for an RF connection request and, when in the second scan mode, scans the predetermined frequency band at a second scan rate for the RF connection request.

18. The IMD of claim 10, wherein the controller, in response to receiving an RF connection request over the predetermined frequency band, causes the RF module to initiate scanning of a secondary frequency band for communications data from an external device.

19. A system comprising:
a patient care system (PCS) configured to monitor at least one physiological attribute of a patient;
an implantable medical device (IMD) that is configured to wirelessly communicate with the PCS, the IMD comprising:
an RF module configured to operate in first and second scan modes;
a sensor configured to sense a posture state of a patient; and
a controller configured to cause the RF module to switch between the first and second scan modes based on the posture state of the patient.

20. The system of claim 19, wherein the controller causes the RF module to operate in the first scan mode when the posture state of the patient is vertical, and wherein the controller causes the RF module to operate in the second scan mode when the posture state of the patient is horizontal.
